(19) United States
(12) Patent Application Publication
(10) Pub. No.: US 2002/0177865 A1
(43) Pub. Date: Nov. 28, 2002

(54) Device and method for forming a hole in a tissue wall of a patient

(75) Inventor: Scott McIntosh, Pacifica, CA (US)

Correspondence Address: BURNS DOANE SWECKER & MATHIS L L P, POST OFFICE BOX 1404, ALEXANDRIA, VA, 22313-1404, US

(73) Assignee: Abbot Laboratories

(21) Appl. No.: 09/732,015

(22) Filed: Dec. 6, 2000

(51) Int. Cl. A61B 17/32

(52) U.S. Cl. 606/184

(57) ABSTRACT

A hole forming device for forming a hole in a vessel wall is provided. The hole forming device comprises a shaft. It further comprises a sheath arranged to be axially advanced along the shaft. It yet further comprises a distal end formation on the shaft. The distal end formation is arranged to pierce and pass through a vessel wall and to cooperate with the sheath, so that when the distal end formation has pierced and has been passed through the vessel wall, and the sheath is then advanced along the shaft, a peripheral portion of the vessel wall is drawn into the sheath and severed from the rest of the vessel wall thereby to form a hole in the vessel wall. Advantageously, the device is arranged to form the hole in a hemostatic manner. A method of forming a hole in a vessel wall is also provided.

### FIELD OF THE INVENTION

This invention relates to forming a hole in a tissue wall in a patient body. In particular, the invention relates to devices and methods for forming a hole in a vessel wall, such as a blood vessel wall, or the like, in a patient body.

### BACKGROUND OF THE INVENTION

During surgical procedures, it is often desired to form a hole in a vessel wall, such as a blood vessel wall, or the like. For example, when a side-to-side anastomosis, or an end-to-side anastomosis, or the like, is to be formed during Coronary Artery Bypass Surgery (CABG), for example, a hole is typically formed in a side of at least one blood vessel so as to permit blood to flow to, or from, that vessel and through the hole after the anastomosis has been performed and the CABG surgery completed.

It is often desired to form a hole in a vessel wall such that the hole has a desired shape. In the case of an end-to-side anastomosis to be formed between an end of one vessel and a side of another vessel, for example, it is often desired to form a hole in the side of the one vessel such that the hole has a generally circular shape.

Traditional methods of forming such a hole in a vessel wall typically involve forming a cut, or incision, in the vessel wall with an instrument, such as scissors, a scalpel, or the like, and then passing an appropriate hole forming device, such as a punch, or the like, through the cut, or incision, so that the punch can then be actuated to form a hole in the vessel wall. This can be the case in a proximal anastomosis, for example.

There are several disadvantages to forming a hole in a vessel wall by first having to form an incision, or cut. One disadvantage is that the hole, when formed, normally does not conform to the shape of hole intended. In particular, the incision tends to cause the shape of the hole to be warped when formed. This is because the incision normally causes an uneven compression distribution around the hole forming device, so that when the hole forming device is actuated to form the hole, the hole is warped due to the uneven compression distribution about the hole forming device. For example, when a hole forming device arranged to form a circular hole is passed through a cut, or incision, and then actuated to form the hole, the hole normally ends up having a generally oval shape.

Another disadvantage of forming a hole in a vessel wall in this way, by first having to form an incision, or cut, is that an instrument, such as a scalpel, scissors, or the like, is typically required in addition to a hole forming device, such as, a punch, or the like. It would be advantageous if a hole could be made in a vessel wall without requiring an additional instrument such as a scalpel, scissors, or the like. In such a case, a medical procedure involving the forming of a hole in a vessel wall should be made simpler than when traditional methods and devices are employed. Furthermore, the hole forming operation should take less time to perform than when traditional methods and devices are used, thereby decreasing the time taken to perform the medical procedure. In certain types of surgery, such as CABG surgery, for example, the time from the commencement of the surgery to its completion should preferably be as little as possible so as to reduce patient trauma. Accordingly, by using a hole forming device which obviates the need for forming an initial incision in the vessel wall with a separate instrument, the forming of anastomoses between blood vessels, or grafts, and/or the like, in a CABG procedure should take less time to perform. In consequence, the CABG procedure as a whole should take less time to perform. This should in turn reduce patient trauma resulting from the CABG procedure.

In the case where the vessel wall is subject to an internal pressure, such as in the case of a blood vessel, or aorta, or the like, another disadvantage of forming a hole in a vessel wall by first forming an incision, or cut, is that clamping of the vessel wall is typically required so as to inhibit leakage of blood from the vessel during the hole forming procedure. In the case where an anastomosis is to be formed at the aorta, for example, the aorta is often cross-clamped. It has been found that such cross-clamping can cause material, such as plaque, or other deposits, or the like, to become dislodged from an inner surface of the wall of the aorta. Such dislodged material can cause neurological problems, which can be fatal. For example, the patient may suffer a stroke, or the like. It would be advantageous if a hole could be formed in a vessel wall under hemostatic conditions. In this way, the need for clamping a pressurized vessel could be done away with. The time taken to form an anastomosis between blood vessels, and/or grafts, or the like, for example, should then further be reduced and the hole forming procedure yet further simplified. Furthermore, the anastomosis procedure should be able to be performed with greater safety in that the risk of dislodging material from an inner surface of the vessel should at least be reduced.

Furthermore, traditional hole forming devices typically have lateral dimensions determined by the size of the hole to be formed in the vessel by the hole forming device. It would be advantageous to be able to provide a hole forming device for forming a hole of the same size as such traditional hole forming devices, but with a device having a reduced lateral dimension. This should enable the device to be used in a less invasive manner thereby further reducing patient trauma. Furthermore, this should enhance the dexterity whereby the device can be used and accessing the vessel wall through overlying patient tissue should be achievable more easily.

### SUMMARY OF THE INVENTION

In accordance with one aspect of the invention, a device and method is provided for forming a hole in a vessel wall without forming an initial incision, and with a device having lateral dimensions less than that of traditional devices used to form a similar sized hole in a vessel wall. Advantageously, the device has an extreme lateral dimension small enough to enable it to be passed through a hemostatic sheath.

Accordingly, there is provided a hole forming device for forming a hole in a vessel wall. The hole forming device comprises a shaft and a sheath arranged to be axially advanced along the shaft. The hole forming device further comprises a distal end formation on the shaft, the distal end formation being arranged to pierce and pass through a vessel wall and to cooperate with the sheath, so that when the distal end formation has pierced and has been passed through the vessel wall, and the sheath is then advanced along the shaft, a peripheral portion of the vessel wall is drawn into the sheath and then severed from the rest of the vessel wall thereby to form a hole in the vessel wall.

By providing the device with a distal end formation on the shaft, which end formation is arranged to pierce and pass through a vessel wall, the use of another instrument, such as a scalpel, or the like, should not be necessary. By providing the device with a sheath arranged to cooperate with the shaft so as to draw a peripheral portion of the vessel wall into the sheath before the peripheral portion is severed, the device should have lateral dimensions less than those of traditional devices used to form a hole of generally the same size.

The distal end formation may define a distal pointed end for piercing the vessel wall. Accordingly, the distal end formation may then be passed through the vessel wall by urging the pointed end against the vessel wall so as to cause it to pierce the vessel wall to form a puncture in the vessel wall and then urging the rest of the distal end formation through the puncture.

Typically, the distal end formation may be arranged to pierce and pass through the vessel wall hemostatically. Accordingly, the device may then typically be used to form a hole in the vessel wall of a vessel while the vessel is under pressure, such as in the case where the vessel is a blood vessel, such as, for example, an aorta, or the like, which is subjected to internal blood pressure.

The distal end formation may define at least one spiral groove extending from the pointed end so that the distal end formation may be caused to pass through the vessel wall by rotating the distal end formation relative to the vessel wall.

The distal end formation may define at least two spiral grooves extending from the pointed end, the one spiral groove extending in a clockwise direction and the other extending in an anti-clockwise direction. Accordingly, the distal end formation may then be rotated either in a clockwise or counter-clockwise direction to cause it to pass through the vessel wall along one of the grooves. Instead, the distal end formation may be rotated in a clockwise and counter-clockwise direction to cause it to pass through the vessel wall along a portion of each of the grooves.

The distal end formation may define a cutting edge positioned proximally relative to the distal end. The cutting edge may be arranged to cooperate with the sheath so as to sever the peripheral portion of the vessel wall when the sheath passes over the cutting edge.

The distal end formation may further define a gripping formation positioned proximally relative to the cutting edge. The gripping formation may be arranged to cooperate with the sheath so that when the sheath is advanced over the gripping formation, the peripheral portion of the vessel wall is gripped between the sheath and the gripping formation thereby to cause the peripheral portion to be drawn into the sheath as the sheath is advanced over the gripping formation.

The sheath may have a cross-sectionally circular shape defining an axially extending internal diameter. The shaft may then be received in an axially extending passage of the sheath defined within the internal diameter, so that the sheath can be caused selectively to advance and retract over the shaft.

The gripping formation may be in the form of a circumferentially extending shoulder on the shaft. The shoulder may define a circumferential edge having an outer diameter. The outer diameter of the circumferential edge may be less than the inner diameter of the sheath. The diameter of the shoulder and the internal diameter of the sheath may typically be sized so that when the sheath passes over the shoulder the peripheral portion of the vessel wall is gripped therebetween.

The cutting edge may extend circumferentially at a diameter generally equal to the internal diameter of the sheath so as to cause the peripheral portion of the vessel wall to be severed by a shearing action as the sheath passes over the cutting edge.

In accordance with another aspect of the invention, there is provided a method of forming a hole in a vessel wall. The method comprises piercing the vessel wall with an end of a distal end formation of a hole forming device so as to form a puncture in the vessel wall. The method further comprises passing the distal end formation of the device through the puncture and then advancing a sheath of the device over the distal end formation thereby to cause a peripheral portion of the vessel wall adjacent the puncture to be drawn into the sheath and then to be severed as the sheath passes over the distal end formation so as to form a hole in the vessel wall.

According to yet another aspect of the invention, there is provided a hole forming device for forming a hole in a vessel wall. The hole forming device comprises a shaft having a distal end and a proximal end, the distal end having a penetrating element to form a puncture, a cutting edge proximal to the penetrating element, and a gripping edge proximal to the cutting edge. The device further comprises a sheath axially translatable over the shaft so as to cause the gripping edge to capture a peripheral portion of the vessel wall and to draw the peripheral portion into the sheath and to cause the cutting edge to sever the peripheral portion of the vessel wall after it has been drawn into the sheath so as to form a hole in the vessel wall having lateral dimensions greater than the cutting edge.

According to yet a further aspect of the invention, there is provided a method of forming a hole in a vessel wall. The method comprises penetrating the vessel wall with a device to form a puncture in the vessel wall, positioning a cutting edge of the device and a gripping edge of the device, positioned proximally of the cutting edge, into a lumen of the vessel adjacent the puncture in the vessel wall and advancing a sheath axially over the gripping edge and the cutting edge so as to capture a peripheral portion of the vessel wall with the gripping edge and to draw the peripheral portion into the sheath so as to sever the peripheral portion with the cutting edge thereby to form a hole in the vessel wall having lateral dimensions greater than the cutting edge.

In accordance with another aspect of the invention, there is provided a method of forming a hole in a vessel wall. The method comprises forming a hole in a vessel wall with a hole forming device, such that the hole forming device extends into the vessel through the hole after the hole has been formed by the hole forming device. The method further comprises advancing a hemostatic sheath along the hole forming device so as to extend into the vessel through the hole, such that the hemostatic sheath extends hemostatically through the hole.

The hole forming device may be arranged to form the hole in the vessel wall hemostatically, forming the hole in the vessel wall with the hole forming device then comprising forming the hole such that after the hole is formed, the hole forming device extends hemostatically through the vessel wall.

The method may comprise passing the hole forming device through the hemostatic sheath prior to forming the hole in the vessel wall with the hole forming device. The hemostatic sheath may have an elongate tubular portion defining a longitudinally extending internal passage and the hole forming device may have an elongate shaft portion. Passing the hole forming device through the hemostatic sheath may then comprise positioning the elongate shaft portion of the hole forming device such that the shaft portion extends internally along the passage of the hemostatic sheath.

Forming the hole in the vessel wall with the hole forming device may comprise forming the hole in the vessel wall with the hole forming device while the elongate shaft portion of the hole forming device extends internally along the passage of the hemostatic sheath.

Advancing the hemostatic sheath along the hole forming device may comprise advancing the hemostatic sheath along the shaft portion of the hole forming device such that an end of the tubular portion of the hemostatic sheath enters the vessel through the hole between an edge of the vessel wall defining the hole and the shaft of the hole forming device.

The method may further comprise withdrawing the hole forming device from the hemostatic sheath after the hemostatic sheath has been passed through the hole.

### BRIEF DESCRIPTION OF THE DRAWINGS

The invention will now be described, by way of example only, with reference to the accompanying diagramatic drawings, in which:

FIG. 1A shows a schematic side view of a hole forming device in accordance with the invention, the device being in a dormant condition;

FIG. 1B shows a schematic side view corresponding to FIG. 1A, the device being in an actuated condition;

FIG. 1C shows a side view corresponding to FIG. 1A, and shows further details of the device;

FIG. 1D shows a schematic view of a locking arrangement of the hole forming device;

FIG. 1E shows a side view of part of the hole forming device in greater detail;

FIGS. 2-7 show steps involved in a method of forming a hole in a vessel wall using the hole forming device of the invention;

FIG. 7B shows a schematic side view of a hole having been formed in the blood vessel wall with the hole forming device of the invention;

FIG. 7C shows a schematic sectional side view of part of a vessel wall having a hole formed with conventional hole forming devices;

FIG. 7D shows a schematic sectional side view of part of a vessel wall having a hole formed with a hole forming device in accordance with the invention;

FIG. 8A shows a schematic three-dimensional view of a preferred distal end portion of a hole forming device in accordance with the invention;

FIG. 8B shows a schematic side view of the distal end portion shown in FIG. 8A;

FIG. 9 shows a schematic end view of part of a distal end portion of another hole forming device in accordance with the invention;

FIG. 10 shows a schematic end view of part of a distal end portion of yet another hole forming device in accordance with the invention;

FIG. 11 shows a schematic end view of part of yet another distal end portion of a hole forming device in accordance with the invention;

FIG. 12A shows a schematic side view of part of a distal end portion of another hole forming device in accordance with the invention;

FIG. 12B shows an end view of the part of the end portion shown in FIG. 12A along arrows XI-XI in FIG. 12A;

FIG. 13A shows a schematic side view of part of an end portion of yet another hole forming device in accordance with the invention;

FIG. 13B shows a schematic end view of the part of the distal end portion shown in FIG. 13A along arrows XII-XII in FIG. 13A;

FIG. 14A shows a schematic side view of part of an end portion of yet another hole forming device in accordance with the invention;

FIG. 14B shows a schematic end view of the part of the distal end portion shown in FIG. 14A along arrows XIII-XIII in FIG. 14A;

FIG. 15 shows a schematic side view of part of an end portion of yet another hole forming device in accordance with the invention;

FIG. 16 shows a schematic side view of part of a distal end portion of yet another hole forming device in accordance with the invention;

FIG. 17 shows a schematic side view of part of another hole forming device in accordance with the invention;

FIG. 18 shows a schematic side view of part of yet another hole forming device in accordance with the invention;

FIG. 19 shows a schematic side view of part of a distal end portion of yet another hole forming device in accordance with the invention;

FIG. 20A shows a schematic three-dimensional view of a hemostatic sheath assembly;

FIG. 20B shows a partial sectional view of part of the hemostatic sheath assembly of FIG. 20B;

FIG. 21 shows a schematic side view of a hole forming device in accordance with the invention extending through part of a sheath of the hemostatic sheath assembly shown in FIGS. 20A and 20B, the hole forming device having been used to form a hole in a vessel wall in a fashion similar to that shown with reference to FIGS. 2 to 7 while extending through the sheath of the hemostatic sheath assembly; and

FIG. 22 shows a schematic side view corresponding to FIG. 21, the hemostatic sheath of the hemostatic sheath assembly having been advanced into a vessel through the hole formed by the hole forming device.

### DESCRIPTION OF THE SPECIFIC EMBODIMENTS

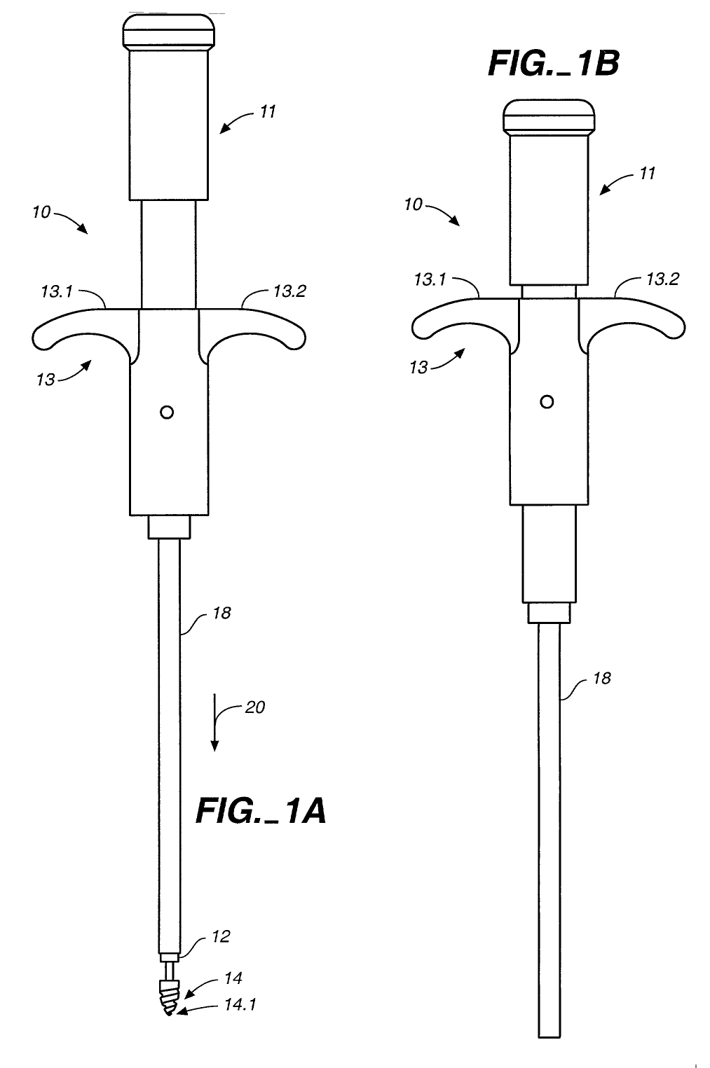

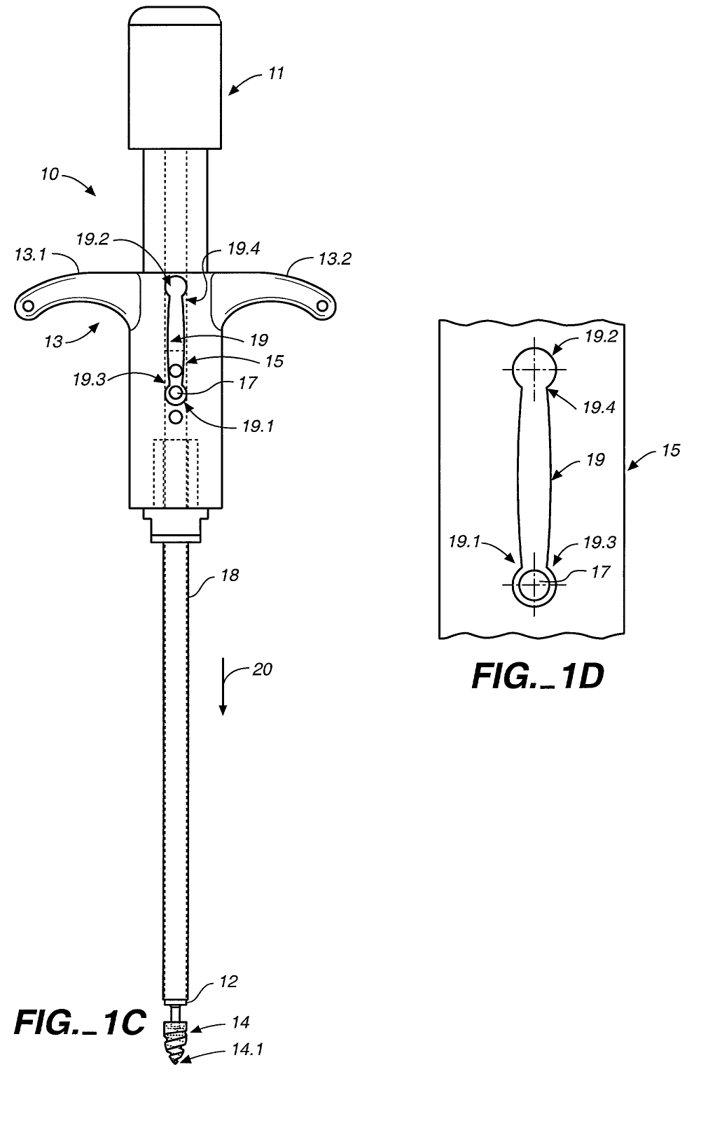

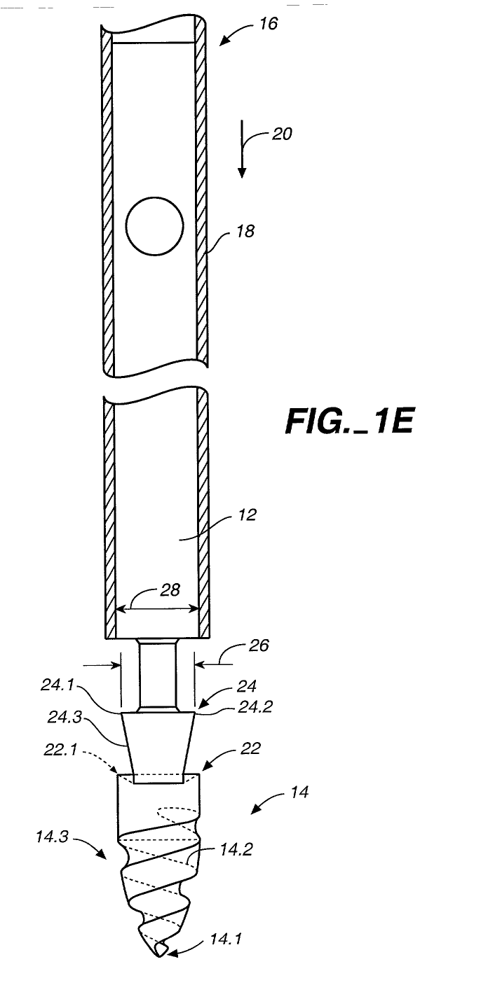

Referring to FIG. 1A and 1B of the drawings, a hole forming device for forming a hole in a vessel wall, in accordance with the invention, is generally indicated by reference numeral 10. In FIG. 1A, the device 10 is shown in a dormant condition, while in FIG. 1B, it is shown in an actuated condition.

Although the invention will be described with reference to using the device 10 to form a hole in a blood vessel wall, such as a wall of an aorta, or the like, it will be appreciated that the invention is not to be limited to forming a hole in a blood vessel wall only, but extends to forming a hole in a tissue wall of a patient body in general.

The device 10 comprises a shaft 12. The shaft 12 has a distal end formation, or portion, generally indicated by reference numeral 14. The shaft 12 further comprises a proximal end generally indicated at 16, as can best be seen with reference to FIG. 1E of the drawings. The distal end formation 14 has an end 14.1 arranged to pierce a vessel wall and to form a puncture in the vessel wall, so that the distal end formation 14 can be passed through the puncture and accordingly also through the vessel wall, as will be described in greater detail hereinbelow.

The device 10 further comprises a sheath 18 mounted on the shaft 12 to be axially displaceable therealong, as indicated by arrow 20. The sheath 18 is arranged to cooperate with the distal end formation 14 so that when the distal end formation 14 of the shaft 12 has been passed through the vessel wall, the sheath 18 can be advanced over the distal end formation 14 thereby to cause a peripheral portion of the vessel wall, adjacent the puncture formed by the distal end 14.1, to be drawn into the sheath 18 and then to be severed so as to form a hole in the vessel wall, as will be described in greater detail hereinbelow.

The device 10 further comprises a plunger arrangement 11 and a finger grip arrangement 13. The finger grip arrangement 13 comprises two opposed limbs 13.1, 13.2. In use, the device 10 is held in a user's hand such that the plunger arrangement 11 abuts against the user's palm, while two fingers are urged against the limbs 13.1, 13.2. To actuate the device 10 from its dormant condition, as shown in FIG. 1A, to its actuated condition, as shown in FIG. 1B, the user urges his or her fingers and his or her palm together thereby to displace the plunger arrangement 11 and the finger grip arrangement 13 toward each other. The shaft 12 is typically operatively connected to the finger grip arrangement 13 and the sheath 18 is typically operatively connected to the plunger arrangement 11. Accordingly, when the plunger arrangement 11 and the finger grip arrangement 13 are displaced toward each other in this fashion, the sheath 18 advances along the shaft 12 and over the distal end formation 14.

Conveniently, and as can best be seen with reference to FIGS. 1C and 1D of the drawings, the device 10 has a locking arrangement 15. The locking arrangement 15 comprises a pin 17 on the shaft 12. It further comprises a slot 19 defined in the sheath 18. The pin 17 rides along the slot 19 when the device 10 is actuated. The slot 19 defines two opposed seats 19.1, 19.2. Each seat 19.1, 19.2 defines a neck formation 19.3, 19.4 for snap-lockingly holding the pin captive in the seats 19.1, 19.2. When the device is in its dormant condition, the pin 17 is held captive in the seat 19.1 by the neck formation 19.3. When the user actuates the device 10, as described above, by causing the plunger arrangement 11 and the finger grip arrangement 13 to displace toward each other thereby to cause the sheath 18 to advance along the shaft 12 and over the distal end formation 14, the pin 17 is caused to ride along the slot 19 from the seat 19.1 and into snap-locking engagement with the seat 19.2, so as to be held captive in the seat 19.2 by the neck formation 19.4. By snap-lockingly holding the pin 17 captive in the seat 19.2 in this fashion, the distal end portion 14.1 is inhibited from inadvertently being advanced from the sheath, thereby inhibiting inadvertent injury, or damage, after a hole has been formed, as described in greater detail herein below. Although, a snap-locking arrangement has been shown, it will be appreciated that any other appropriate locking arrangement can be used instead.

As can best be seen in FIG. 1E of the drawings, the end 14.1 is typically pointed. The distal end formation 14 comprises a rearwardly-tapering surface, or formation, 14.2 tapering outwardly and in an operatively rearward direction from the end 14.1. At least one spiral groove 14.3 extends along the surface 14.2 from the pointed end 14.1.

A cutting edge, positioned proximally relative to the end 14.1 is indicated by reference numeral 22. The cutting edge 22 is arranged to cooperate with the sheath 18 so as to sever a peripheral portion of a vessel wall therebetween when the sheath 18 is advanced over the cutting edge 22. The cutting edge 22 typically defines an adjacent undercut formation 22.1. Although the cutting edge 22 is shown as extending circumferentially in a straight fashion, it will be appreciated that the cutting edge can be in any appropriate form. For example, instead of extending circumferentially in a straight fashion, the cutting edge can be serrated, or the like.

The distal end formation 14 further defines a gripping formation generally indicated at 24. The gripping formation 24 is positioned proximally of the cutting edge 22. The gripping formation 24 is arranged to cooperate with the sheath 18 so as to grip the peripheral portion of the vessel wall therebetween thereby to cause the peripheral portion to be drawn into the sheath 18 as the sheath 18 is advanced over the gripping formation 24. The gripping formation 24 typically defines a circumferentially extending shoulder 24.1 on the shaft 18. The shoulder 24.1 defines a circumferential edge 24.2. The circumferential edge 24.2 has an outer diameter 26 less than an inner diameter 28 of the sheath 18, as can best be seen in FIG. 1E. The diameter 26 of the circumferential edge 24.2 is typically between about 1.5 mm and about 3.5 mm less than the internal diameter 28 of the sheath, when the device 10 is arranged to form a hole in a typical blood vessel.

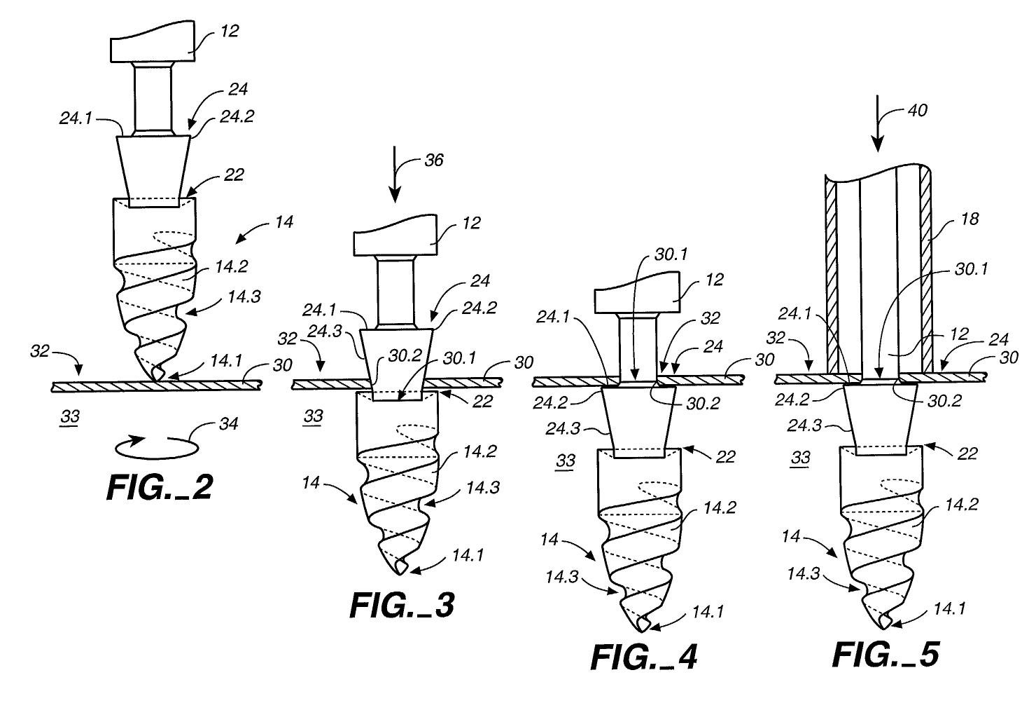

Forming a hole in a blood vessel wall with the device 10 will now be described with reference to FIGS. 2 to 7 of the drawings, in which like reference numerals have been used to designate similar parts and features unless otherwise stated.

Referring initially to FIG. 2, the end 14.1 is urged against a blood vessel wall 30 of a blood vessel 32. The shaft 12 is then caused to rotate relative to the vessel wall 30 thereby to cause the distal end formation 14 to rotate, as indicated by arrow 34. As the shaft 12 rotates in this fashion, the pointed end 14.1 pierces and punctures the wall 30 to form a puncture 30.1 in the vessel wall 30, as can best be understood with reference to FIG. 3 of the drawings. As the shaft 12 is rotated further, a peripheral edge 30.2, of the vessel wall 30, adjacent the puncture 30.1, rides along the tapering surface 14.2 in the spiral groove 14.3 until the peripheral edge 30.2 drops behind the surface 14.2 as indicated in FIG. 3. By causing the peripheral edge to ride along the surface 14.2 in the groove 14.3 in this fashion, the formation 14 can be passed through the vessel wall in a relatively controlled manner. Therefore, the risk of pushing the distal end formation 14 through the vessel wall too far, so that it punctures the vessel wall at an opposed side, for example, can be inhibited. Accordingly, the formation 14 can be passed through the vessel wall in a relatively safe manner.

It will be appreciated that as the peripheral edge 30.2 of the vessel wall 30 adjacent the puncture 30.1 rides along the tapering surface 14.2 in the spiral groove 14.3, the peripheral edge 30.2 is tensioned around the surface 14.2 and in the groove 14.3 thereby permitting hemostatic passage through the vessel wall 30.

After the vessel wall 30 has dropped behind the tapering surface 14.2, the shaft 12 is urged in the direction of arrow 36, as indicated in FIG. 3, until the vessel wall drops behind the shoulder formation 24, as can best be seen in FIG. 4 of the drawings. Typically, a smooth tapering surface 24.3 extends from behind the surface 14.2 to the shoulder formation 24 so that when the shaft is urged in the direction of arrow 36, the vessel wall hemostatically rides over the surface 24.3 until it drops behind the shoulder formation 24, as can best be seen with reference to FIG. 4 of the drawings. Advantageously, the shaft 12 is caused to displace angularly to and fro in a clockwise and counter clockwise direction to assist in causing the vessel wall 30 to ride along the tapering surface 24.3.

Referring now to FIG. 5, when the distal end formation 14 has been inserted through the vessel wall 30, and typically into a lumen 33 of the vessel 32 defined within the wall 30, the device 10 is actuated, as described above, by the user urging his or her fingers and his or her palm together thereby to displace the plunger arrangement 11 and the finger grip arrangement 13 toward each other. In this fashion, the sheath 18 is advanced along the shaft 12, as indicated by arrow 40, until it abuts against the vessel wall 30.

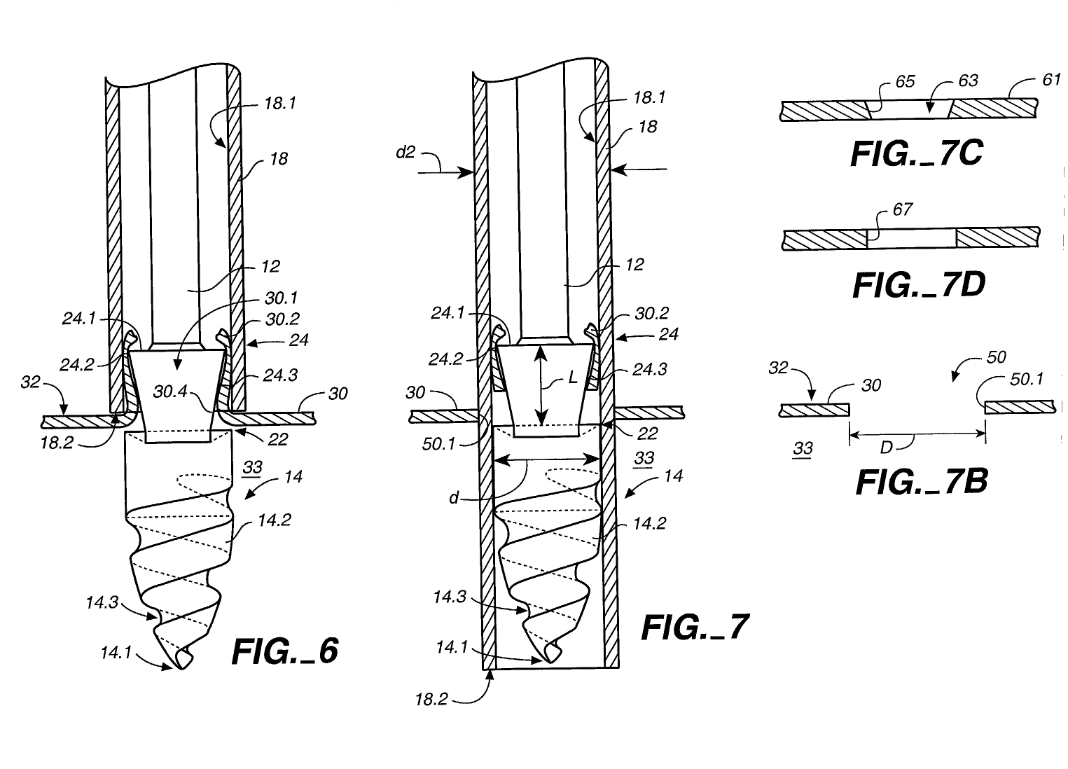

As the sheath 18 is advanced further in the direction of arrow 40, as indicated in FIG. 6 of the drawings, a peripheral portion 30.4 of the vessel wall 30, immediately adjacent the edge 30.2, is gripped between the gripping formation 24 and an inner surface 18.1 of the sheath 18, so that when the sheath 18 is advanced further, the peripheral portion 30.4 is drawn into the sheath 18. As the peripheral portion 30.4 is drawn into the sheath 18 in this fashion, it is typically placed in tension, or stretched, between a leading circumferential edge 18.2 of the sheath 18 and the gripping formation 24.

As the sheath 18 is advanced yet further, it passes over the cutting edge 22, as can best be seen in FIG. 7 of the drawings. As it passes over the cutting edge 22, the peripheral portion 30.4 is severed between the cutting edge 22 and the sheath 18 as the leading edge 18.2 of the sheath 18 passes over the cutting edge 22. It will be appreciated that as the sheath 18 was advanced along the shaft 12 in this fashion, the pin 17 rode along the slot 19 from a position in the seat 19.1 to a position in the seat 19.2. Since the pin is then held captive in the seat 19.2 by the neck formation 19.4, the pointed end 14.1 is inhibited from accidentally being extended from the sheath 18. In this fashion, inadvertent piercing or other damage to the patient is inhibited since the pointed end is retained in the sheath 18 after the hole has been formed in the vessel wall.

After the peripheral portion 30.4 has been severed in this fashion, a hole 50 is formed in the vessel wall 30, as can best be seen with reference to FIG. 7B of the drawings. It will be appreciated that since the peripheral portion 30.4 was stretched as it was drawn into the sheath 18 and before it was severed, the hole 50 has a diameter D greater than a diameter d of the cutting edge 22. It will be appreciated that the diameter D of the hole 50 to be made in the vessel wall 30 can be varied by varying a distance L between the gripping formation 24 and the cutting edge 22. Advantageously, the device 10 is arranged so as to form a hole having a diameter D which is greater than the diameter d of the cutting edge 22 but less than an outer diameter d2 of the sheath 18 so that after the hole 50 has been formed, an edge 50.1 of the vessel 30 adjacent the hole 50 hemostatically embraces the sheath 18, as can best be seen in FIG. 7.

With reference to FIG. 7C of the drawings, a hole formed in a vessel wall 61 with a conventional hole forming device, or punch, is generally indicated by reference numeral 63. It has been found that when such conventional hole forming devices, or punches, are used, the hole formed is defined by an inclined circumferential edge 65. Such a hole 63 can unnecessarily hinder blood flow in the case where the hole is formed during an anastomosis procedure. When the hole forming device in accordance with the invention is used, the circumferential edge is closer to being perpendicular relative to the wall of the vessel, as can best be seen with reference to FIG. 7D, and as indicated by reference numeral 67.

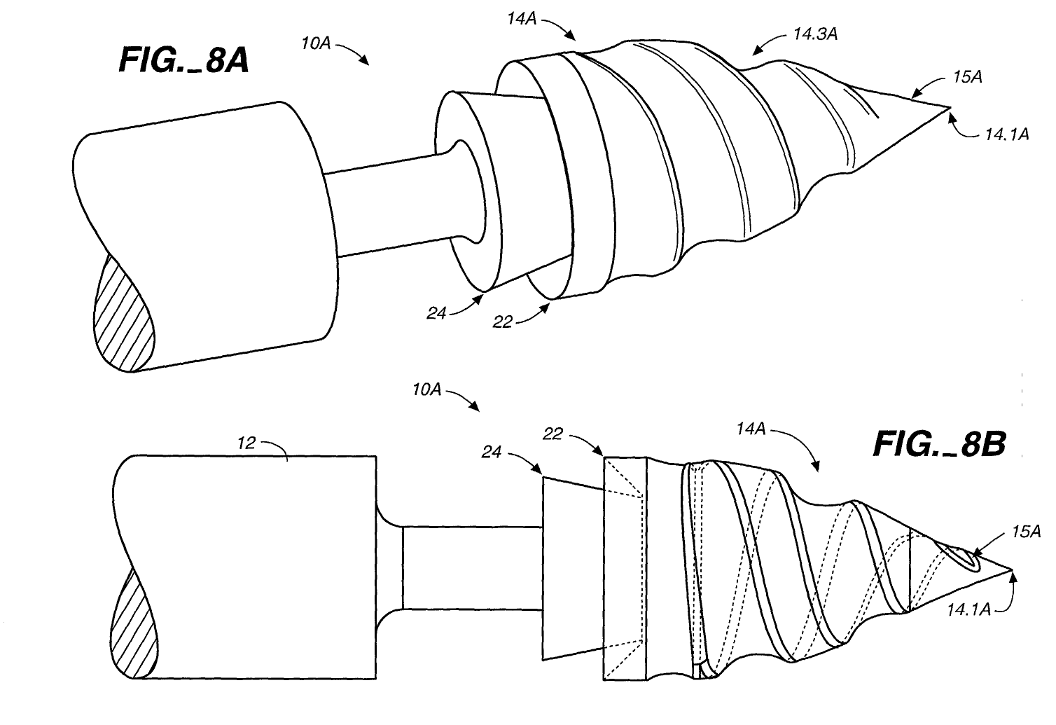

Referring now to FIGS. 8A and 8B, a preferred distal end portion of a device 10A in accordance with the invention, is generally indicated by reference numeral 14A. In FIGS. 8A and 8B similar reference numerals have been used to designate similar parts, or features, unless otherwise stated.

The distal end portion 14A is similar to the portion 14 described above, but has been formed to inhibit, or at least reduce, twisting of the vessel wall 30 in response to the distal end portion 14A being displaced angularly relative to the vessel wall 30 to pierce and pass through the vessel wall 30. To this end, the helical groove 14.3A is in the form of a smooth, relatively shallow, and gradually rounded groove, so as to inhibit, or at least reduce, friction between the portion 14A and the vessel wall. Accordingly, the vessel wall is inhibited from being “grabbed” by the portion 14A as the portion 14A passes therethrough. As indicated at 15A, the groove 14.3A starts at a position operatively rearwardly of the point 14.1A so as to inhibit, or at least reduce, twisting of the vessel wall 30 when the portion 14A is used to pierce the vessel wall.

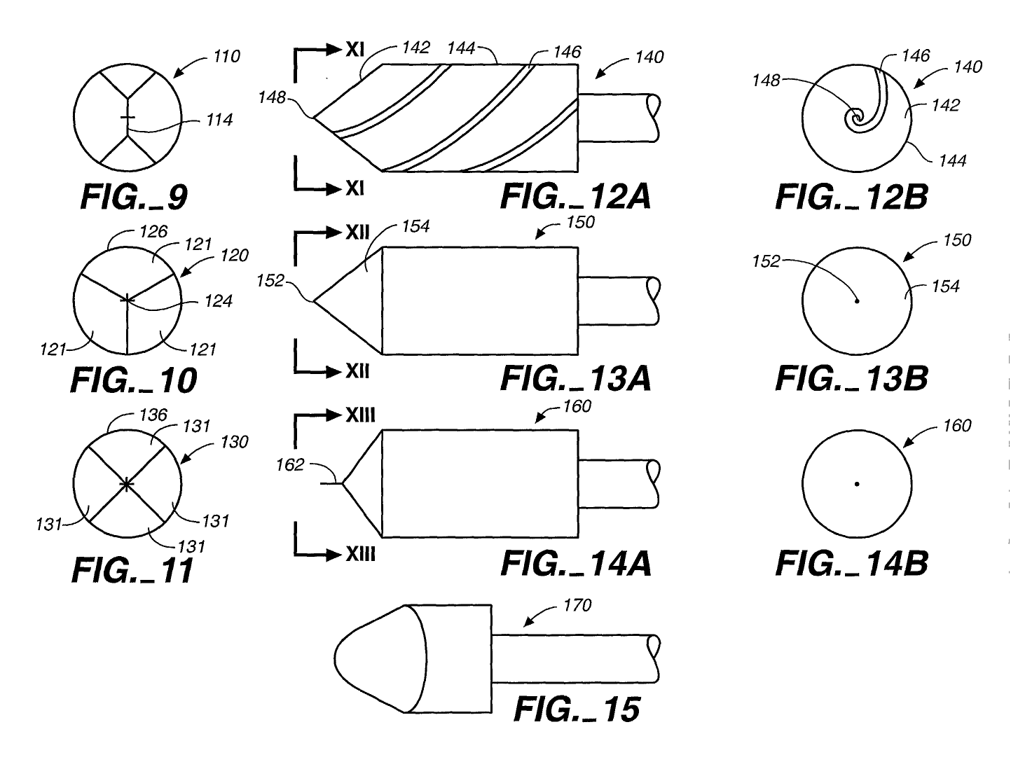

Referring to FIG. 9 of the drawings, part of another hole forming device for forming a hole in a vessel wall, is generally indicated by reference numeral 110. The device 110 is similar to the device 10 save that its tapering formation 112 has a sharp cutting edge 114 for penetrating through a vessel wall. Such an arrangement can be beneficial where the device is to be used to form a hole in a vessel wall which has a grain, such as the aorta, or the like. In such a case, the cutting edge 114 can be positioned to extend generally perpendicularly relative to the grain when piercing the aorta thereby to inhibit, or at least reduce, the risk of tearing the aorta wall.

Referring to FIG. 10 of the drawings, a tapering formation of another hole forming device in accordance with the invention is generally indicated by reference numeral 120. The tapering formation 120 has three faces 121 which extend from a pointed end 124 to a generally circular cutting edge at 126. The pointed end 124 is arranged to penetrate a vessel wall by urging the pointed end therethrough.

A tapering formation of another hole forming device in accordance with the invention is generally indicated by reference numeral 130 in FIG. 11. The tapering formation 130 of the device shown in FIG. 11 is similar to the tapering formation 120 shown in FIG. 10, save that it has four faces 131 which extend from its pointed end to a generally circular cutting edge at 136.

Referring now to FIGS. 12A and 12B of the drawings, a distal end formation of yet another hole forming device in accordance with the invention is generally indicated by reference numeral 140. The distal end formation 140 comprises a tapering portion at 142 and a constant diameter portion 144 extending from the tapering portion 142. The distal end formation 140 further comprises a helical groove 146 leading from an end 148.

Referring to FIGS. 13A and 13B of the drawings, yet another distal end formation of a hole forming device in accordance with the invention is generally indicated by reference numeral 150. The distal end formation 150 is similar to the distal end formation shown in FIGS. 12A and 12B, save that it does not have a helically extending groove formation 146 extending along its tapering portion 154. Accordingly, in use, the distal end formation 150 is urged through the vessel wall with or without rotation relative to the vessel wall, so that a leading end 152 pierces the vessel wall and the vessel wall rides directly up and over the tapering portion 154.

Referring to FIGS. 14A and 14B of the drawings, a distal end formation of yet another hole forming device in accordance with the invention is generally indicated by reference numeral 160. Generally, the distal end formation 160 is used in a fashion similar to the distal end formation 150 of FIGS. 13A and 13B, save that the distal end formation 160 has a pin formation 162 arranged to pierce the vessel wall.

Referring now to FIG. 15 of the drawings, a distal end formation of yet another hole forming device in accordance with the invention is generally indicated by reference numeral 170. In use, the distal end formation 170 is inserted through a puncture, aperture, or the like, in the vessel wall, to permit the distal end formation 170 to be passed therethrough.

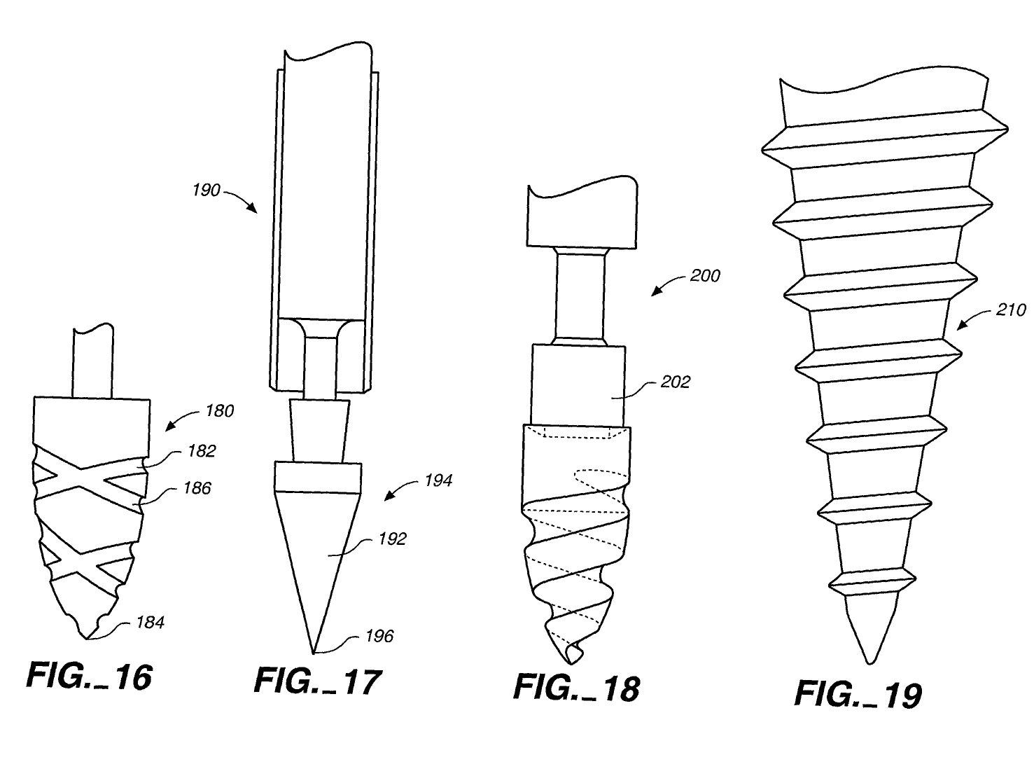

Referring to FIG. 16 of the drawings, a distal end formation of another hole forming device in accordance with the invention is generally indicated by reference numeral 180. The distal end formation 180 is similar to the distal end formation 14 of the device shown in FIGS. 1-7, save that the distal end formation 180 has two grooves extending helically from its end 184. The one groove, indicated by reference numeral 182, extends in a generally clockwise direction, and the other groove, indicated by reference numeral 186, extends in a generally counter-clockwise direction. Accordingly, in use, the distal end formation can be rotated in either a clockwise or a counter-clockwise direction so as to cause the vessel wall to ride up the distal end formation 180 in one of the grooves 182, 186. Instead, the distal end formation can be angularly displaced to and fro in both a clockwise and a counter-clockwise direction so as to cause the vessel wall to ride up the distal end formation 180 along portions of both of the grooves 182, 186.

Referring now to FIG. 17 of the drawings, another hole forming device in accordance with the invention is generally indicated by reference numeral 190. The hole forming device 190 is similar to the hole forming device 10 shown in FIGS. 1-7, save that its distal end formation 194 defines a smooth tapering surface 192 leading to a pointed end 196. In use, the formation 194 is pushed against the vessel wall to cause the leading end 196 to pierce the vessel wall to permit the distal end formation 194 to be passed directly through the vessel wall. Advantageously, the formation 194 can be displaced angularly to and fro in a clockwise and counter-clockwise direction to ease passage through the vessel wall and to reduce insertion force so as to enable the formation 194 to be passed through the vessel wall in a relatively controlled manner. In this fashion, the risk of pushing the distal end formation 194 through the vessel wall too far so that it punctures the vessel wall at an opposed side of the vessel wall, for example, can be inhibited.

Referring now to FIG. 18 of the drawings, a distal end formation of yet another hole forming device in accordance with the invention is generally indicated by reference numeral 200. The distal end formation 200 is similar to the distal end formation 14 of the device 10 shown in FIGS. 1-7, save that the surface 202 extending from the cutting edge to the shoulder formation extends at a constant diameter and does not taper as in the case of the device 10.

Referring now to FIG. 19 of the drawings, yet another distal end formation of another hole forming device in accordance with the invention is generally indicated by reference numeral 210. The distal end formation 210 comprises a tapering threaded formation for puncturing the vessel wall and for causing the vessel wall to ride screw-threadedly therealong.

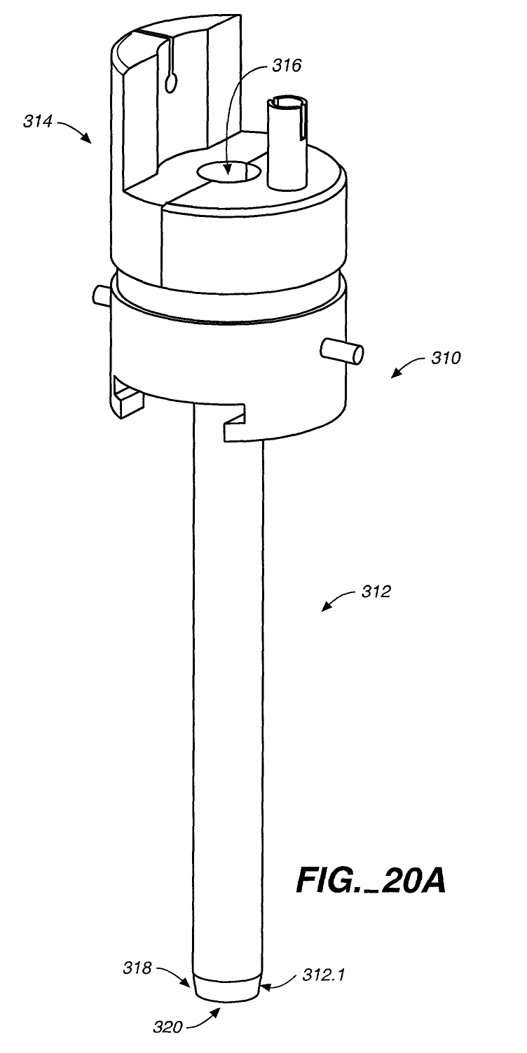

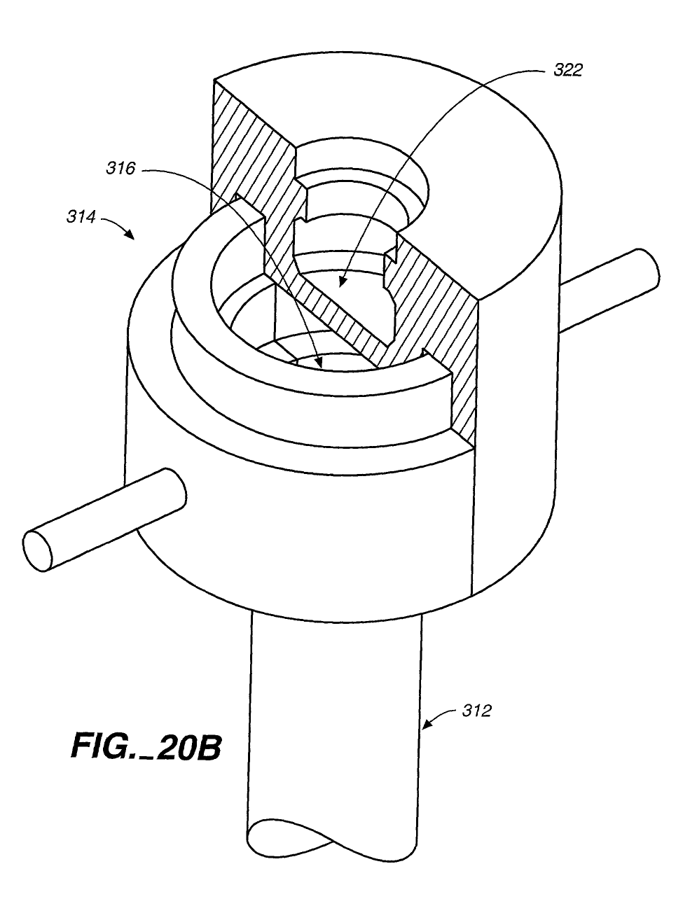

Referring to FIGS. 20A and 20B, a hemostatic sheath assembly is generally indicated by reference numeral 310. The assembly 310 comprises a hemostatic sheath portion, or elongate tubular portion, 312 and a valve body 314. The sheath portion 312 is elongate and defines a longitudinally extending passage 316 and a free end 318 defining an open mouth 320 leading into the passage 316. The valve body 314 is mounted at an opposed end of the elongate sheath portion 312.

As can best be seen with reference to FIG. 20B, the valve body 314 defines a seal formation 322. The seal formation 322 is arranged sealingly to close the opposed end of the sheath portion 312. However, the seal formation 322 is arranged to permit passage of an instrument therethrough. In use, the assembly 310 can be passed through a hole in a blood vessel, or the like, so that the free end 318 is positioned within the vessel, the vessel wall then sealingly engaging the sheath portion 312 so that the sheath portion 312 is hemostatically received by the vessel. The mouth 320 is then positioned in the vessel and the seal formation 322 inhibits blood from passing therethrough. Different instruments can then be selectively introduced into the vessel by passing the instruments through the seal formation, the seal formation inhibiting the escape of blood from the vessel when the instruments are selectively passed into the vessel through the seal formation.

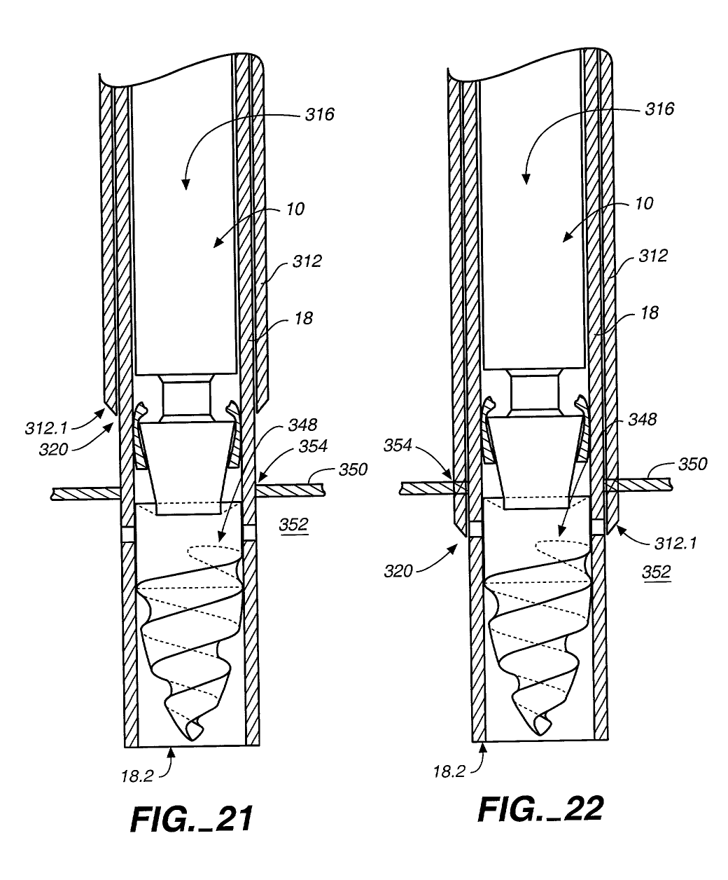

In accordance with another aspect of the invention, a method of forming a hole in a vessel wall will now be described with reference to FIGS. 21 and 22. In FIGS. 21 and 22, the same reference numerals as used herein above, have been used to designate similar parts or features unless otherwise stated.

Referring initially to FIG. 21, the hole forming device 10 shown in FIGS. 1 to 7 above, is shown in a condition in which its sheath 18 has been positioned to extend internally along the hemostatic sheath portion 312 of the hemostatic sheath assembly 310 shown in FIGS. 20A and 20B. Accordingly, the hemostatic sheath assembly 310 is positioned such that it extends along the sheath 18 of the device 10. The sheath 18 extends co-axially within and along the passage 316 and through the seal formation 322, not shown in FIGS. 21 and 22, of the hemostatic sheath assembly 310. The seal formation 322 sealingly embraces the sheath 18.

As shown in FIG. 21, after the hemostatic sheath assembly has been so positioned on the sheath 18 of the device 10, the device 10 is used to form a hole 348 in a wall 350 of a vessel 352 in a fashion similar to that described above with reference to FIGS. 2 to 7 of the drawings. After the hole 348 has been formed by the device 10, the device 10 is urged carefully in an operatively forward direction to cause the end 18.2 of the sheath 18 to pass through the hole 348. After the end 18.2 has been urged through the hole 348 in this fashion, an edge 354 of the vessel wall 350 defining the hole 348 hemostatically seals against an end portion of the sheath 18 at the end 18.2. Accordingly, by using the device 10 the hole 348 is formed under hemostatic conditions.

After the hole 348 has been formed by the device 10, and as can best be seen with reference to FIG. 22 of the drawings, the hemostatic sheath assembly 310 is advanced along the sheath 18 of the device 10 such that the end 320 passes through the hole 348 between the sheath 18 and the edge 354 of the vessel wall 350 defining the hole 348. Advantageously, an end portion 312.1 of the hemostatic sheath portion 312 is tapered in an operatively rearward direction to assist in passing the end 320 of the hemostatic sheath portion 312 through the hole 348 and to ease the edge 354 of the wall 350 radially outwardly. After the end 320 has been passed through the hole 348 in this fashion, the edge 354 of the wall 350 hemostatically embraces the hemostatic sheath portion 312.

After the hemostatic sheath assembly 310 has been so positioned to extend through the hole 348, the device 10 can be removed from the hemostatic sheath assembly 310 by withdrawing the device 10 through the seal formation 322. In this way the hemostatic sheath assembly 310 can be operatively positioned to extend through a hole in a blood vessel, or aorta, or the like, under hemostatic conditions. After the device 10 has been removed from the hemostatic sheath assembly 310, the assembly 310 can be left in place relative to the vessel 352 to serve as a port through which instruments can be passed to access the vessel 352. Although a method of forming a hole in a vessel wall is described above with reference to FIGS. 21 and 22 using the device 10 of the invention, it will be appreciated that any other appropriate hole forming device can be used instead of the device 10. For example, any one of the hole forming devices described above with reference to FIGS. 8 to 19 can be used instead. Furthermore, any other appropriate hole forming device, or punch, could be used in the method as described with reference to FIGS. 21 and 22. For example, a conventional hole forming device, or punch, can be used, such that the method can then comprise forming an initial incision in the vessel wall, passing the conventional hole forming device through the incision while it extends through a hemostatic sheath assembly, and then advancing the hemostatic sheath assembly relative to the hole forming device so as to pass through the hole in the vessel wall in a manner similar to that described above with reference to FIGS. 21 and 22. When such a conventional hole forming device is used in this fashion, the method can include clamping the blood vessel for a short period so as to inhibit blood from escaping from the vessel from when the incision is made to when the hemostatic sheath assembly has been hemostatically positioned relative to the blood vessel.

Although certain embodiments of the invention have been described above in detail for purposes of clarity and understanding, it will be appreciated that the invention has been described with reference to the above embodiments by way of example only, and that modifications or changes can be made without detracting from the essence of the invention. Accordingly, the scope of the invention is to be defined by the appended claims with due regard to equivalents of the claimed elements or features.

What is claimed is:

1. A hole forming device for forming a hole in a vessel wall, the hole forming device comprising:
a shaft;
a sheath arranged to be axially advanced along the shaft; and
a distal end formation on the shaft, the distal end formation being arranged to pierce and pass through a vessel wall and to cooperate with the sheath, so that when the distal end formation has pierced and has been passed through the vessel wall, and the sheath is then advanced along the shaft, a peripheral portion of the vessel wall is drawn into the sheath and severed from the rest of the vessel wall thereby to form a hole in the vessel wall.

2. The hole forming device of claim 1, wherein the distal end formation defines a distal pointed end for piercing the vessel wall.

3. The hole forming device of claim 2, wherein the distal end formation is arranged to pierce and pass through the vessel wall hemostatically.

4. The hole forming device of claim 3, wherein the distal end formation defines at least one spiral groove extending from the pointed end so that the distal end formation can be caused to pass through the vessel wall by rotating the distal end formation relative to the vessel wall.

5. The hole forming device of claim 4, wherein the distal end formation defines at least two spiral grooves extending from the pointed end, the one spiral groove extending in a clockwise direction and the other extending in an anti-clockwise direction.

6. The hole forming device of claim 2, wherein the distal end formation defines a cutting edge positioned proximally relative to the distal end, the cutting edge being arranged to cooperate with the sheath so as to sever the peripheral portion of the vessel wall when the sheath passes over the cutting edge.

7. The hole forming device of claim 6, wherein the distal end formation defines an under-cut formation adjacent the cutting edge.

8. The hole forming device of claim 6, wherein the distal end formation further defines a gripping formation positioned proximally relative to the cutting edge, the gripping formation being arranged to cooperate with the sheath so that when the sheath is advanced over the gripping formation the peripheral portion of the vessel wall is gripped between the sheath and the gripping formation thereby to cause the peripheral portion to be drawn into the sheath as the sheath is advanced over the gripping formation.

9. The hole forming device of claim 8, wherein the sheath has a cross-sectionally circular shape and defines an axially extending internal diameter.

10. The hole forming device of claim 9, wherein the gripping formation is in the form of a circumferentially extending shoulder on the shaft, the shoulder defining a circumferential edge having an outer diameter, the outer diameter of the circumferential edge being less than the inner diameter of the sheath.

11. The hole forming device of claim 10, wherein the diameter of the circumferential edge is between about 3.5 mm and about 3.5 mm less than the internal diameter of the sheath.

12. The hole forming device of claim 9, wherein the cutting edge extends circumferentially at a diameter generally equal to the internal diameter of the sheath so as to cause the peripheral portion of the vessel wall to be severed by a shearing action as the sheath passes over the cutting edge.

13. A method of forming a hole in a vessel wall, the method comprising:
piercing a vessel wall with an end of a distal end formation of a hole forming device so as to form a puncture in the vessel wall;
passing the distal end formation of the device through the puncture; and
advancing a sheath of the device over the distal end formation to cause a peripheral portion of the vessel wall adjacent the puncture to be drawn into the sheath and then to be severed as the sheath passes over the distal end formation, thereby to form a hole in the vessel wall.

14. The method of claim 13, which comprises stretching the peripheral portion of the vessel wall as it is drawn into the sheath and before it is severed.

15. The method of claim 14, wherein stretching the peripheral portion of the vessel wall comprises gripping the peripheral portion between the distal end formation and the sheath as the sheath is advanced over the distal end formation.

16. The method of claim 13, wherein severing the peripheral portion of the vessel wall comprises shearing the peripheral portion between the sheath and the distal end formation.

17. The method of claim 13, wherein the hole forming device comprises a pointed end and piercing the vessel wall comprises passing the pointed end through the vessel wall to form the puncture.

18. The method of claim 17, wherein passing the distal end formation through the puncture in the vessel wall comprises causing a peripheral edge of the vessel wall adjacent the puncture to ride along a surface of the distal end formation, which surface extends from the pointed end.

19. The method of claim 18, wherein the surface is arranged to permit the edge of the vessel wall adjacent the puncture to ride along the surface hemostatically.

20. The method of claim 19, wherein causing the peripheral edge of the vessel wall adjacent the puncture to ride along the surface of the distal end formation comprises rotating the distal end formation relative to the vessel wall thereby to cause the vessel wall to ride along a groove on the distal end formation.

21. A method of forming a hole in a vessel wall, the method comprising:
forming a hole in a vessel wall with a hole forming device, such that the hole forming device extends into the vessel through the hole after the hole has been formed by the hole forming device; and
advancing a hemostatic sheath along the hole forming device so as to extend into the vessel through the hole, such that the hemostatic sheath extends hemostatically through the hole.

22. The method of claim 21, wherein the hole forming device is arranged to form the hole in the vessel wall hemostatically, forming the hole in the vessel wall with the hole forming device comprising forming the hole such that after the hole is formed, the hole forming device extends hemostatically through the vessel wall.

23. The method of claim 22, which comprises passing the hole forming device through the hemostatic sheath prior to forming the hole in the vessel wall with the hole forming device.

24. The method of claim 23, wherein the hemostatic sheath has an elongate tubular portion defining a longitudinally extending internal passage and the hole forming device has an elongate shaft portion, passing the hole forming device through the hemostatic sheath comprising positioning the elongate shaft portion of the hole forming device such that the shaft portion extends internally along the passage of the hemostatic sheath.

25. The method of claim 24, wherein forming the hole in the vessel wall with the hole forming device comprises forming the hole in the vessel wall with the hole forming device while the elongate shaft portion of the hole forming device extends internally along the passage of the hemostatic sheath.

26. The method of claim 25, wherein advancing the hemostatic sheath along the hole forming device comprises advancing the hemostatic sheath along the shaft portion of the hole forming device such that an end of the tubular portion of the hemostatic sheath enters the vessel through the hole between an edge of the vessel wall defining the hole and the shaft of the hole forming device.

27. The method of claim 22, which further comprises withdrawing the hole forming device from the hemostatic sheath after the hemostatic sheath has been passed through the hole.

28. A hole forming device for forming a hole in a vessel wall, the hole forming device comprising:
a shaft having a distal end and a proximal end, the distal end having a penetrating element to form a puncture, a cutting edge proximal to the penetrating element, and a gripping edge proximal to the cutting edge; and
a sheath axially translatable over the shaft so as to cause the gripping edge to capture a peripheral portion of the vessel wall and to draw the peripheral portion into the sheath and to cause the cutting edge to sever the peripheral portion of the vessel wall after it has been drawn into the sheath so as to form a hole in the vessel wall having lateral dimensions greater than the cutting edge.

29. A method of forming a hole in a vessel wall, the method comprising:
penetrating a vessel wall with a device to form a puncture in the vessel wall;
positioning a cutting edge of the device and a gripping edge of the device, positioned proximally of cutting edge, into a lumen of the vessel adjacent the puncture in the vessel wall; and
advancing a sheath axially over the gripping edge and the cutting edge so as to capture a peripheral portion of the vessel wall with the gripping edge and to draw the peripheral portion into the sheath so as to sever the peripheral portion with the cutting edge thereby to form a hole in the vessel wall having lateral dimensions greater than the cutting edge.
